(19) United States
(12) Patent Application Publication
(10) Pub. No.: US 2019/0003977 A1
(43) Pub. Date: Jan. 3, 2019

(54) ANALYTE SENSING DEVICE

(71) Applicant: AVAGO TECHNOLOGIES GENERAL IP (SINGAPORE) PTE. LTD., Singapore (SG)

(72) Inventors: James Costello, Singapore (SG); Boon Keat Tan, Singapore (SG); Serene Chan, Singapore (SG); Wee Sin Tan, Singapore (SG)

(21) Appl. No.: 15/785,909

(22) Filed: Oct. 17, 2017

Related U.S. Application Data
(60) Provisional application No. 62/527,750, filed on Jun. 30, 2017.

(51) Int. Cl. G01N 21/78; A61B 5/08; A61B 5/1477; A61B 5/00; G01N 33/52

(52) U.S. Cl. CPC G01N 21/78; A61B 5/082; G01N 33/525; A61B 5/6898; A61B 5/1477

(57) ABSTRACT

An analyte sensing device and a mobile device incorporating an analyte sensing device are disclosed. One example of an analyte sensing device is disclosed to include a body, a sensor die, and a substantially transparent material positioned such that the sensor die is sandwiched between the body and the substantially transparent material. The sensor die may be in optical communication with the substantially transparent material and in electrical communication with the body.

### CROSS REFERENCE TO RELATED APPLICATIONS

Pursuant to 35 U.S.C. § 119(e), the present application claims the benefit of and priority to U.S. Provisional Application Ser. No. 62/527,750, filed on Jun. 30, 2017, the entire disclosure of which is hereby incorporated by reference, in its entirety, for all that it teaches and for all purposes.

### FIELD OF THE DISCLOSURE

Example embodiments are generally directed toward sensors and devices incorporating the same and more specifically toward an analyte sensing device.

### BACKGROUND

A biosensor is a device used for the detection of an analyte (e.g., a substance whose chemical constituents are being identified and measured), that combines a biological component with a physicochemical detector. The sensitive biological element (e.g., tissue, microorganisms, organelles, cell receptors, enzymes, antibodies, nucleic acids, etc.) is usually a biologically derived material or biomimetic component that interacts (e.g., binds or recognizes) with the analyte under study. The detector element of the biosensor transforms the signal resulting from the interaction of the analyte with the biological element into another signal (e.g., an electrical signal) that can be more easily measured, quantified, and/or processed by a microprocessor or similar circuit. The detector element can utilize any type of transducer (e.g., an optical transducer, a piezoelectric, an electrochemical transducer, etc.). While biosensors are known, most, if not all, biosensors are incorporated into purpose-built devices that are highly immobile or inconvenient for their users.

### BRIEF DESCRIPTION OF THE DRAWINGS

Inventive concepts are described in conjunction with the appended figures, which are not necessarily drawn to scale:

FIG. 1 is a schematic block diagram depicting a mobile device in accordance with at least some embodiments of the present disclosure;

FIG. 2 is a block diagram depicting details of an analyte sensing device in accordance with at least some embodiments of the present disclosure;

FIG. 3A is a block diagram depicting a first construction of components of an analyte sensing device in accordance with at least some embodiments of the present disclosure;

FIG. 3B is a block diagram depicting a second construction of components of an analyte sensing device in accordance with at least some embodiments of the present disclosure;

FIG. 3C is a block diagram depicting a third construction of components of an analyte sensing device in accordance with at least some embodiments of the present disclosure;

FIG. 4A is an isometric view of an analyte sensing device in accordance with at least some embodiments of the present disclosure;

FIG. 4B is a cross-sectional view of the analyte sensing device depicted in FIG. 4A;

FIG. 5A is an isometric view of another analyte sensing device in accordance with at least some embodiments of the present disclosure;

FIG. 5B is a cross-sectional view of the analyte sensing device depicted in FIG. 5A;

FIG. 6A is a top view of a chemochromic layer for an analyte sensing device in accordance with at least some embodiments of the present disclosure;

FIG. 6B is an isometric view depicting a chemochromic layer relative to a set of detectors in accordance with at least some embodiments of the present disclosure;

FIG. 6C is a top view depicting a first configuration of a chemochromic layer relative to a set of detectors in accordance with at least some embodiments of the present disclosure;

FIG. 6D is a top view depicting a second configuration of a chemochromic layer relative to a set of detectors in accordance with at least some embodiments of the present disclosure;

FIG. 7A is a waveform illustrating a first spectral profile in accordance with at least some embodiments of the present disclosure;

FIG. 7B is a waveform illustrating a second spectral profile in accordance with at least some embodiments of the present disclosure;

FIG. 7C is a waveform illustrating a third spectral profile in accordance with at least some embodiments of the present disclosure;

FIG. 7D is a waveform illustrating a first transmission profile of a chemochromic material in a first state in accordance with at least some embodiments of the present disclosure;

FIG. 7E is a waveform illustrating a second transmission profile of a chemochromic material in a second state in accordance with at least some embodiments of the present disclosure;

FIG. 7F is a waveform illustrating a first spectral response in accordance with at least some embodiments of the present disclosure;

FIG. 7G is a waveform illustrating a second spectral response in accordance with at least some embodiments of the present disclosure;

FIG. 7H is a waveform illustrating a third spectral response in accordance with at least some embodiments of the present disclosure;

FIG. 8 is a cross-sectional view of an alternative design of an analyte sensing device in accordance with at least some embodiments of the present disclosure;

FIG. 9 is an isometric view of yet another alternative design of an analyte sensing device in accordance with at least some embodiments of the present disclosure;

FIG. 10 is an isometric view of yet another alternative design of an analyte sensing device in accordance with at least some embodiments of the present disclosure;

FIG. 11A is an isometric view of an analyte sensing device in accordance with at least some embodiments of the present disclosure;

FIG. 11B is an isometric view of an alternative configuration for the analyte sensing device depicted in FIG. 11A;

FIG. 11C is a cross-sectional view of an analyte sensing device as shown in either FIG. 11A or 11B;

FIG. 12A is an isometric view of another analyte sensing device in accordance with at least some embodiments of the present disclosure;

FIG. 12B is an isometric view of an alternative configuration for the analyte sensing device depicted in FIG. 12A;

FIG. 12C is a cross-sectional view of an analyte sensing device as shown in either FIG. 12A or 12B;

FIG. 13A is a cross-sectional view of a single molded analyte sensing device in accordance with at least some embodiments of the present disclosure;

FIG. 13B is a cross-sectional view of a double molded analyte sensing device in accordance with at least some embodiments of the present disclosure;

FIG. 14 is a cross-sectional view of a portion of a mobile device incorporating a chemochromic layer in accordance with at least some embodiments of the present disclosure;

FIG. 15 is a cross-sectional view of a portion of a mobile device configured to detect analytes through a cavity in accordance with at least some embodiments of the present disclosure;

FIG. 16A is a cross-sectional view of an analyte sensing device having a wire-bonded package in accordance with at least some embodiments of the present disclosure; and

FIG. 16B is a cross-sectional view of an analyte sensing device having a flip-chip package in accordance with at least some embodiments of the present disclosure.

### DETAILED DESCRIPTION

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of example embodiments will be described herein with reference to drawings that are schematic illustrations of idealized configurations. As such, variations from the shapes of the illustrations as a result, for example, manufacturing techniques and/or tolerances, are to be expected. Thus, the various aspects of example embodiments presented throughout this document should not be construed as limited to the particular shapes of elements (e.g., regions, layers, sections, substrates, etc.) illustrated and described herein but are to include deviations in shapes that result, for example, from manufacturing. By way of example, an element illustrated or described as a rectangle may have rounded or curved features and/or a gradient concentration at its edges rather than a discrete change from one element to another. Thus, the elements illustrated in the drawings are schematic in nature and their shapes are not intended to illustrate the precise shape of an element and are not intended to limit the scope of example embodiments.

It will be understood that when an element such as a region, layer, section, substrate, or the like, is referred to as being “on” another element, it can be directly on the other element or intervening elements may also be present. In contrast, when an element is referred to as being “directly on” another element, there are no intervening elements present. It will be further understood that when an element is referred to as being “formed” or “established” on another element, it can be grown, deposited, etched, attached, connected, coupled, or otherwise prepared or fabricated on the other element or an intervening element.

Furthermore, relative terms, such as “lower” or “bottom” and “upper” or “top” may be used herein to describe one element's relationship to another element as illustrated in the drawings. It will be understood that relative terms are intended to encompass different orientations of an apparatus in addition to the orientation depicted in the drawings. By way of example, if an apparatus in the drawings is turned over, elements described as being on the “lower” side of other elements would then be oriented on the “upper” side of the other elements. The term “lower” can, therefore, encompass both an orientation of “lower” and “upper” depending of the particular orientation of the apparatus. Similarly, if an apparatus in the drawing is turned over, elements described as “below” or “beneath” other elements would then be oriented “above” the other elements. The terms “below” or “beneath” can therefore encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

As used herein, the singular forms “a,” “an,” and “the” are intended to include the plural forms as well, unless the context clearly indicates otherwise. It will be further understood that the terms “include,” “includes,” “including,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

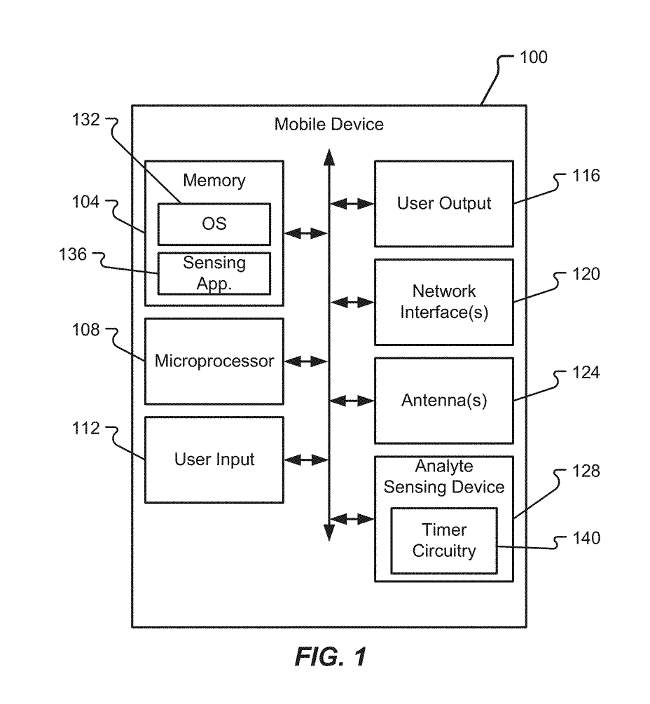

Referring now to FIGS. 1-16B, various configurations of analyte sensing devices and mobile devices having analyte sensing devices incorporated therein will be described. In some embodiments, an analyte sensing device may be incorporated into a mobile device, such as, for example, a mobile phone, a wearable device, a portable computer, or a tablet. The disclosure is not limited with respect to the types of devices or systems in which the analyte sensing device of this disclosure are used. Furthermore, the analyte sensing device as disclosed in this disclosure may be provided in wafer level, chip level, package level, or combinations thereof.

Analyte, as used herein, may be in the form of solid particles, liquid, gel, gas, droplets or other forms. Generally, the package for the analyte sensing device may be separated in two types. The first type has a flat surface for direct contact with the analyte which may be a portion of a human body, for example. The second type has a cavity that is in fluid communication with the analyte. The second type of analyte sensing device may be suitable to detect droplets from breath when a user blows into the cavity.

Further, in this disclosure, the term “light” or “radiation” may be interpreted as a specific type of electro-magnetic wave. Alternatively or additionally, “light” or “radiation” may be interpreted to include all variations of electro-magnetic waves. For example, ultra-violet, infrared, near infrared, and other invisible (to the human eye) radiation may be included when considering the term “light” or “radiation.”

With reference now to FIG. 1, an illustrative mobile device 100 incorporating an analyte sensing device 128 will be described in accordance with at least some embodiments of the present disclosure. In the depicted embodiment, the mobile device 100 is shown in accordance with embodiments of the present disclosure. The mobile device 100 may include one or more components, such as, a memory 104, a microprocessor 108, an antenna(s) 124, a network interface(s) 120, one or more user input 112, and one or more user output 116. In some embodiments, the mobile device 100 may further include a power module. As can be appreciated, the mobile device 100 may be configured to exchange information/data with other mobile devices 100 either via a direct machine-to-machine communication or through a communication network 104.

The memory 104 of the mobile device 100 may be used in connection with the execution of application programming or instructions by the microprocessor 108, and for the temporary or long term storage of program instructions and/or data. The memory 104 may contain executable functions that are used by the microprocessor 108 to run other components of the mobile device 100. In one embodiment, the memory 104 may be configured to store credential information, information related to an electronic ID (e.g., pictures, Personally Identifiable Information (PII), etc.). For instance, the credential information or electronic ID information may include, but is not limited to, unique identifications, names, birthdates, ID expiration dates, addresses, manufacturer identification, passwords, keys, encryption schemes, transmission protocols, and the like. In some embodiments, the memory 104 may be configured to store configuration information, identification information, authentication information, and/or the like. In some embodiments, the memory 104 may comprise volatile or non-volatile memory and a controller for the same. Non-limiting examples of memory 104 that may be utilized in the mobile device 100 include RAM, ROM, buffer memory, flash memory, solid-state memory, or variants thereof.

The microprocessor 108 may correspond to one or many microprocessors that are contained within the housing of the mobile device 100 with the memory 104. In some embodiments, the microprocessor 108 incorporates the functions of the user device's Central Processing Unit (CPU) on a single Integrated Circuit (IC) or a few IC chips. The microprocessor 108 may be a multipurpose, programmable device that accepts digital data as input, processes the digital data according to instructions stored in its internal memory, and provides results as output. The microprocessor 108 implements sequential digital logic as it has internal memory. As with most known microprocessors, the microprocessor 108 may operate on numbers and symbols represented in the binary numeral system.

The one or more antenna(s) 124 may be configured to enable wireless communications between the mobile device 100 and other mobiles devices and/or with a communication network. As can be appreciated, the antenna(s) 124 may be arranged to operate using one or more wireless communication protocols and operating frequencies including, but not limited to, Bluetooth®, NFC, Zig-Bee, GSM, CDMA, WiFi, RF, and the like. By way of example, the antenna(s) 124 may be RF antenna(s), and as such, may transmit RF signals through free-space to be received by a network access point (e.g., a WiFi access point, a cellular tower, etc.). One or more of the antennas 124 may be driven or operated by a dedicated antenna driver.

In some embodiments, the mobile device 100 may include a power module. The power module may be configured to provide power to the parts of the mobile device 100 in order to operate. The power module may store power in a capacitor of the power module. In one embodiment, electronics in the power module may store energy in the capacitor and turn off when an RF field is present. This arrangement can ensure that energy is presented to the mobile device 100 minimizing any effect on read distance. Although the mobile device 100 may be configured to receive power passively from an electrical field of another mobile device 100, it should be appreciated that the mobile device 100 may provide its own power. For example, the power module may include a battery or other power source to supply power to parts of the mobile device 100. The power module may include a built-in power supply (e.g., battery) and/or a power converter that facilitates the conversion of externally-supplied AC power into DC power that is used to power the various components of the mobile device 100. In some embodiments, the power module may also include some implementation of surge protection circuitry to protect the components of the mobile device 100 from power surges.

The mobile device 100 may include a network interface(s) 120 that is configured to communicate with one or more different systems or devices either remotely or locally to the mobile device 100. Thus, the network interface(s) 120 can send or receive messages to or from other devices 100, a network access point, or the like. In some embodiments, the communicated information may be provided to, or exchanged with, other components within the mobile device 100.

The user input 112 may include at least one device sensor. Among other things, a device sensor may be configured to detect a state of the mobile device 100 or location of the mobile device 100. One type of suitable sensor that can be included in the mobile device 100, although not depicted, is a location sensor. A location sensor may be configured to determine a geographical location and/or position of the mobile device 100. In one embodiment, this location may be based on Global Positioning System (GPS) data provided by a GPS module of the mobile device 100.

In some embodiments, the mobile device 100 may include a user interface. The user interface may or may not include one or more of a user input 112 and/or user output 116. Examples of suitable user input 112 devices that may be included in the user interface include, without limitation, buttons, keyboards, mouse, touch-sensitive surfaces, pen, camera, microphone, etc. Examples of suitable user output 116 devices that may be included in the user interface include, without limitation, display screens, touchscreens, lights, speakers, etc. It should be appreciated that the user interface may also include a combined user input 112 and user output 116 device, such as a touch-sensitive display or the like.

As mentioned above, one or more of the antenna(s) 124 may correspond to a communication network interface whereas others of the antenna(s) 124 may correspond to a wireless machine interface. A wireless machine interface may include a Bluetooth interface (e.g., antenna and associated circuitry), an NFC interface (e.g., an antenna and associated circuitry), an Infrared interface (e.g., LED, photodiode, and associated circuitry), and/or an Ultrasonic interface (e.g., speaker, microphone, and associated circuitry). A communication network interface, on the other hand, may include a Wi-Fi/802.11N interface (e.g., an antenna and associated circuitry), an Ethernet port, a Network Interface Card (NIC), a cellular interface (e.g., antenna, filters, and associated circuitry), or the like. The network interface may be configured to facilitate a connection between the mobile device 100 and a communication network and may further be configured to encode and decode communications (e.g., packets) according to a protocol utilized by the communication network 104.

The analyte sensing device 128 is shown to be a part of the mobile device 100. It should be appreciated that the analyte sensing device 128 may be integrated as part of the mobile device 100 or it may be a separate device that is connectable to the mobile device 100. The analyte sensing device 128 may be operated, at least partially, by a sensing application 136 stored in memory 104. As can be appreciated, instructions stored in memory 104 may be executed by the microprocessor 108 or some other IC chip in the mobile device 100. The sensing application 136 may be accessed by a user via the operating system (OS) 132, which is also stored in memory 104. The sensing application 136 may correspond to a specific application (e.g., set of instructions) that facilitate the operation of the analyte sensing device 128. Specifically, the sensing application 136 may include instructions that, when executed by the processor 108, enable outputs of the analyte sensing device 128 to be displayed via the user output 116 and further enable inputs received at the user input 112 to control operation of the sensing application 136 and/or the analyte sensing device 128.

In some embodiments, the analyte sensing device 128 may include circuitry, such as timer circuitry 140, that enables the analyte sensing device 128 to control a particular timing with which the analyte sensing device 128 operates. For instance, the timer circuitry 140 may control an amount of time (e.g., a time period) during which the analyte sensing device 128 is analyzing a chemochromic material and its reaction to an analyte. Said another way, the timer circuitry 140 may control timing operations of the analyte sensing device 128 and may further control an amount of time during which particular analysis operations are performed.

It should be appreciated that the timer circuitry 140 may be separate from the analyte sensing device 128. For instance, timer circuitry (e.g., a clock function) within the microprocessor 108 may be used to replicate the timer circuitry 140. Alternatively or additionally, the microprocessor 108 may provide other circuitry that facilitates operation of the analyte sensing device 128 within the mobile device 100. As a non-limiting example, the microprocessor 108 or some other IC chip within the mobile device 100 may provide a circuit configured to electrically connect a predetermined set of detectors in the analyte sensing device 128 such that the set of detectors provide an output that corresponds with particular chemochromic portions in the analyte sensing device 128. This functionality will be described in further detail herein. It should be appreciated, however, that circuitry enabling operation of the analyte sensing device 128 can be integrated into the analyte sensing device 128 (e.g., an IC chip packed with other components of the analyte sensing device 128) or separated from the analyte sensing device 128 and provided by the microprocessor 108, for instance.

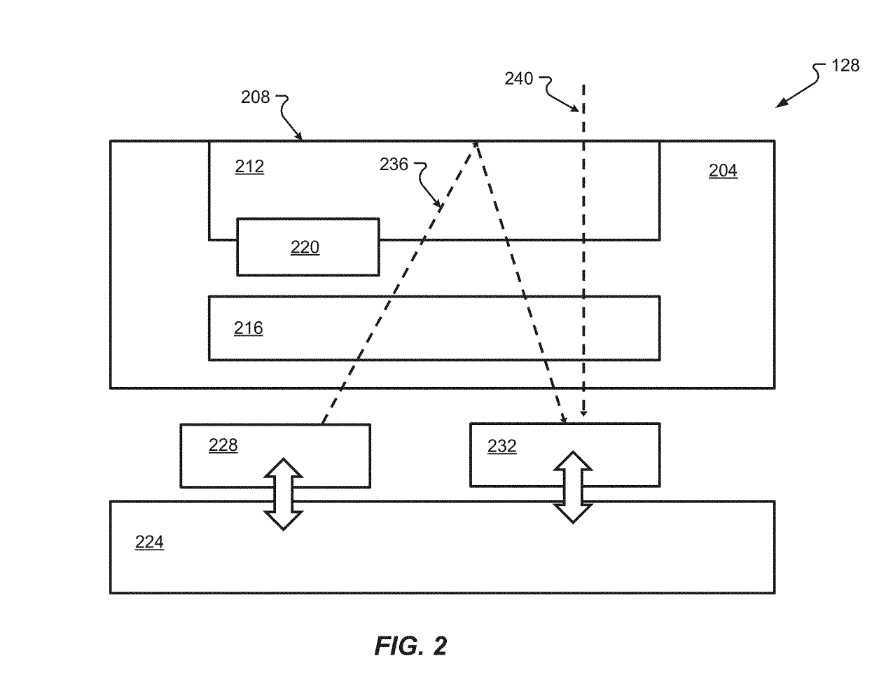

With reference now to FIG. 2, additional details of an analyte sensing device 128 will be described in accordance with at least some embodiments of the present disclosure. The analyte sensing device 128 is shown to include a substantially transparent material 204, a chemochromic material 212, an optical element 216, an interlocking structure 220, a package body 224, an emitter 228, and a sensor die 232. The substantially transparent material 204 is further shown to include a detection surface 208 that is exposed at an external surface of the analyte sensing device 128, thereby enabling the chemochromic material 212 to be directly exposed to an analyte being tested or analyzed.

The emitter 228 is shown to produce an emitted light 236 that is directed through the optical element 216 toward the chemochromic material 212, which may also be referred to herein as a chemochromic layer. At least some of the emitted light 236 may reflect from the chemochromic material 212 and be detected at the sensor die 232. In some embodiments, ambient light 240 may also be present and may pass through the substantially transparent material 204. The ambient light 240 may also be detected at the sensor die 232. In some embodiments, the sensor die 232 may be configured to output an electrical signal indicative of the light received at the sensing surface thereof. In some embodiments, the electrical signal output by the sensor die 232 may include information representing both the emitted light 236 that has reflected off the chemochromic material 212 and the ambient light 240. One or more cancellation algorithms or protocols may be used to separate the portion of the electrical signal produced by the ambient light 240 from the portion of the electrical signal produced by the reflected emitted light 236. In some embodiments, the emitter 228 is an optional component, in which case the chemochromic material 212 is solely illuminated by the ambient light 240.

The substantially transparent material 204 is positioned such that the sensor die 232 is sandwiched between the body 224 and the substantially transparent material 204. The sensor die 232, in some embodiments, is in an optical communication with the substantially transparent material 204 and in an electrical communication with the body 224. More specifically, the body 224 may include one or more Integrated Circuit (IC) components that are electrically connected to the sensor die 232 via one or more wire bonds and/or one or more solder bumps (e.g., via a flip-chip connection).

The substantially transparent material 204 comprises the detection surface 208 which is exposed externally (e.g., away from other components of the analyte sensing device 128) such that the detection surface 208 is adaptable to be in direct contact with the one or more analytes. The substantially transparent material 204 further comprises a chemochromic material 212 or multiple chemochromic materials 212 disposed at least partially adjacent to the detection surface 208 such that a portion of the chemochromic material 212 is configured to be exposed to an analyte via the detection surface 208. The chemochromic material 212, in some embodiments, exhibits a first color in a first state, and a second color in a second state when exposed to a predetermined analyte. It should be appreciated that the chemochromic material 212 may assume more than two states (e.g., turn a third color when exposed to a different analyte), but the concept of a chemochromic material 212 assuming two different colors in two different states will be discussed for ease of understanding embodiments of the present disclosure.

The sensor die 232 may correspond to an IC chip having a photosensitive surface or photodetector provided thereon. In some embodiments, the sensor die 232 may include an array of photodetectors that are configured to convert received electromagnetic energy into an electrical signal. Alternatively or additionally, the sensor die 232 may include a simple photodetector (e.g. a photodiode) or an array of simple photodetectors connected to one another via underlying circuitry in the sensor die 232. In some embodiments, the sensor die 232 is configured to detect the change in color of the chemochromic material 212. The chemochromic material 212, the detection surface 208, and the substantially transparent material 204, in some embodiments, may be integrally formed in a semiconductor package. The analyte sensing device 208 may optionally comprise the emitter 228. The emitter 228 is arranged such that the optical signal emitted from the emitter 112 (e.g., the emitted light 236) is directed to the substantially transparent material 204 so as to be reflected toward the sensor die 232 by the detection surface 208 after passing through the chemochromic material 212.

The optical element 216, as will be described in further detail herein, may correspond to one or multiple elements capable of carrying and/or directing optical signals. Non-limiting examples of an optical element 216 include a lens, a plurality of lenses, a light guide, a plurality of light guides, an optical filter, a film, a mirror, a prism, or combinations thereof.

The interlocking structure 220 is provided as a component that assists with the attachment or integration of the chemochromic material 212 with the substantially transparent material 204. The interlocking structure 220 may be a mechanical structure, an adhesive, a tape, or combinations thereof.

The emitter 228 may correspond to any type of device configured to produce emitted light 236 in response to receiving an electrical signal (e.g., via circuitry in the body 224). Non-limiting example of an emitter 228 include a Light Emitting Diode (LED), an array of LEDs, a laser, a Vertical Cavity Surface Emitting Laser (VCSEL), or combinations thereof.

The body 224 may correspond to a simple substrate. Alternatively or additionally, the body 224 may include one or more electrical traces or connections. Alternatively or additionally, the body 224 may include a semiconductor material (e.g., a semiconductor die) or a package surrounding a semiconductor die (e.g., a plastic housing or the like).

In order to fit into a mobile device 100, the analyte sensing device 128 should be in a small form factor. Providing all elements (e.g., body 224, sensor die 232, substantially transparent material 204, detection surface 208, chemochromic material 212, emitter 228, etc.) into a single miniaturized semiconductor package for mobile devices 100 may be challenging for several reasons. Firstly, the chemochromic material 212 should be externally exposed and may wear out or deteriorate easily when exposed to external environmental conditions. Secondly, having a small form factor device may result in alignment and reliability issues, such as delamination or peeling between components. Thirdly, having a small form factor device also means less light 240 will pass through the chemochromic material 212 to the sensor die. In other words, the sensor die 232 has to have a high sensitivity to work effectively.

There are several ways to incorporate the chemochromic material 212 into a single semiconductor package. However, usually the chemochromic material 212 is integrated (formed as a single unitary unit or as a component together with the substantially transparent material 204). To enable color detection, the chemochromic material 212 and the substantially transparent material 204 are arranged in the optical path of the sensor die 232. The chemochromic material 212 may include organic or inorganic particles. In some embodiments, the particles of the chemochromic material 212 possess the characteristic of changing color when exposed to certain known substances appearing in air, liquid, or solid form. The chemochromic material 212 may comprise a plurality of chemochromic particles, which may be the same or different (e.g., to detect different types of analytes). One or more chemochromic particles may exhibit color change in response to exposure to an analyte. By having a selected set of chemochromic particles to form a chemochromic material 212, the chemochromic material 212 may be adapted to detect one, two, three, four, or more analytes.

The substantially transparent material 204, is some embodiments, is configured to cover and protect the sensor die 232 as well as other conductive traces on a surface of the body 224. The substantially transparent material 204 may be an encapsulant such as an epoxy or silicone configured to encapsulate the sensor die 232. In other embodiments, the substantially transparent material 204 may cover the exposed portion of the sensor die 232 as a lid. In yet another embodiment, the substantially transparent material 204 may be a layer sealing the semiconductor package such that the sensor die 232 is protected within a cavity. The substantially transparent material 204 may be formed as a layer providing structural support to the chemochromic material 212, which is formed as a layer on the substantially transparent material 204 in various exemplary forms.

There are many ways to integrate the substantially transparent material 204 and the chemochromic material 212. The different approaches may work for different types of analyte sensing devices 128 or may be used for specific considerations. The chemochromic material 212 may comprise a plurality of chemical particles in order to respond to more than one analyte. For example, the chemochromic material 212 may comprise organic or inorganic chemical substances diluted in a carrier solvent. The carrier solvent, like the substantially transparent material 204, may be in liquid form during the manufacturing process, but casted or molded into solid form after the manufacturing process. The carrier solvent may be, more suitably but not limited to, a polymer base material for organic chemochromic substances. The adhesion between the carrier solvent and the substantially transparent material may be a consideration for reliability performance.

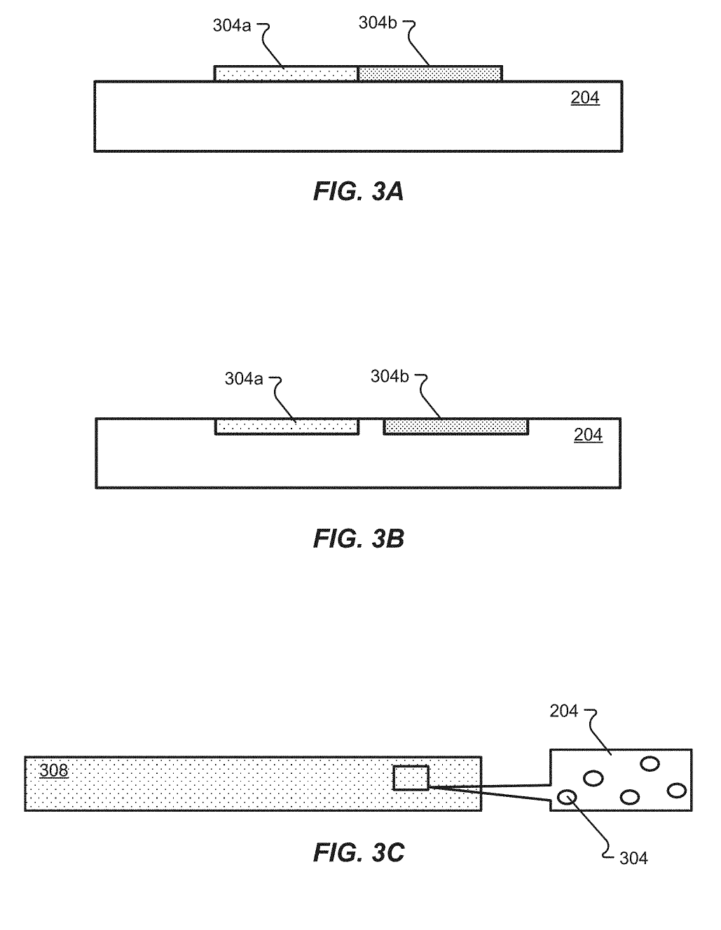

With reference now to FIGS. 3A-C, various configurations of how the substantially transparent material 204 and the chemochromic material 212 may be integrally formed will be described in accordance with at least some embodiments of the present disclosure. In the embodiment of the first construction shown in FIG. 3A, the chemochromic material 212 is shown to include a first chemochromic material 304a and a second chemochromic material 304b deposited as a chemochromic layer on a top surface of the substantially transparent material 204. In some embodiments, the chemochromic layer formed by the chemochromic materials 304a, 304b may be a thin chemochromic layer disposed, printed, coated, laminated, or using other suitable technique to form on the substantially transparent material 204, which is pre-formed or pre-manufactured as a substantially transparent layer having a relatively consistent/constant thickness. In some embodiments, the substantially transparent material 204 is formed as a layer to provide structural support to the chemochromic layer/material 304a, 304b. The chemochromic layer formed by the chemochromic materials 304a, 304b may be provided as a thin layer to the thickness of the substantially transparent layer 204. In one embodiment, the chemochromic material 304a, 304b may be less than 20% the thickness of the substantially transparent layer 204. In another embodiment, the chemochromic material 304a, 304b may be less than 5% the thickness of the substantially transparent layer 204. Interlocking structures 220 may be employed to improve the mechanical interlock or interface between the chemochromic material 304a, 304b and the substantially transparent material 204.

The particular construction depicted in FIG. 3A may be suitable for sensing devices 128 having one or more types of chemochromic material in which the chemochromic material are arranged in a plurality of chemochromic portions such as in an array or in a two-dimensional manner (e.g., in a row or columnar format). As a non-limiting example, the structure of FIG. 3A may be suitable for chemochromic materials 304a, 304b in a powdered form. In addition, this depicted structure may be suitable for chemochromic material(s) 304a, 304b that can be formed thin enough to allow light to pass through while simultaneously demonstrating changes of color.

Alternatively or additionally, the substantially transparent material 204 and the chemochromic material 212 may be integrally formed with one another. More specifically, FIG. 3B depicts an arrangement whereby the first and second chemochromic materials 304a, 304b are provided within the substantially transparent material 204 as opposed to being formed on top of the substantially transparent material 204. In this arrangement, the detection surface (e.g., the top surface of the substantially transparent material) is substantially smooth or flat because the top surface of the chemochromic material(s) 304a, 304b is substantially co-planar with the top surface of the substantially transparent material 204. It should be appreciated that this particular type of integration may help to further avoid delamination between the substantially transparent material 204 and the chemochromic materials 304a, 304b.

In a further alternate embodiment, the substantially transparent material and the chemochromic material may be completely integrated to form a single chemochromic layer 308. In other words, the substantially transparent material 204 may be employed as the carrier solvent for the chemochromic material 304 as illustrated in FIG. 3C. This particular configuration may further help prevent delamination because the particles of the chemochromic material 304 are completely dispersed throughout the substantially transparent material 204. The chemochromic layer 308 formed by this integration may be have a substantially constant thickness or width.

Although specific constructions illustrated in FIGS. 3A-C may correlate with various specific analyte sensing devices 128 described herein, it should be appreciated that the analyte sensing device 128 may be formed using a different construction. For example, the embodiment shown in FIG. 2 may have a construction of integrally formed chemochromic material 212 and substantially transparent material illustrated in any of FIGS. 3A-C and other methods not illustrated above with minor modification as deemed suitable by a person having ordinary skill in the art.

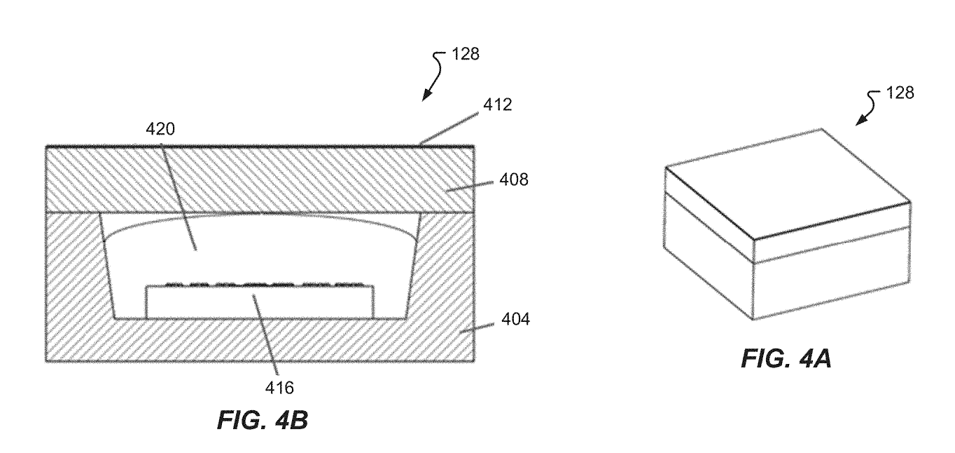

With reference now to FIGS. 4A-B, a specific configuration of an analyte sensing device 128 will be described in accordance with at least some embodiments of the present disclosure. The analyte sensing device 128 is shown to be a version of the analyte sensing device 128 that senses a single analyte. It should be appreciated, however, that the analyte sensing device 128 may be modified to sense more than one analyte. The analyte sensing device 128 is shown to include a body 404 and a sensor die 416 disposed on a receiving surface of the body 404. The body 404, for example, may include a ceramic-based package substrate having a predetermined form. Other suitable substrate materials may also be useful such as polymers, encapsulants, etc. The body 404, in one embodiment, comprises a cavity which is generally enclosed or concealed. The cavity of the body 404, for example, is defined by at least one sidewall and the receiving surface of the body.

As shown, the analyte sensing device 128 also includes a substantially transparent layer 408. The substantially transparent layer 408 is disposed on the body 404 such that the sensor die 416 is positioned between the receiving surface of the body 404 and the substantially transparent layer 408. The analyte sensing device 128 further includes a chemochromic layer 412 disposed on an externally exposed surface of the substantially transparent layer 408. Specifically, the externally exposed surface of the substantially transparent layer 408 may correspond to a surface of the layer 408 that opposes the surface interfacing with the body 404 and facing the sensor die 416. Exposure of the chemochromic layer 412 on the external surface of the substantially transparent layer 408 enables the chemochromic layer 412 to be exposed to external environmental conditions as well as one or more analytes. Meanwhile, the cavity of the body 404 and the body 404 itself protects the sensor die 416 from the same environmental conditions that could adversely impact the sensor die 416. In some embodiments, the chemochromic layer 412 exhibits a first color in a first state (e.g., before exposure to a predetermined analyte), and a second color in a second state (e.g., after exposure to the predetermined analyte). The sensor die 416, in one embodiment, is configured to detect the change in color of the chemochromic layer 412.

The substantially transparent layer 408, for example, includes a glass material, a mold compound, an acrylic material, or other suitable material which is substantially transparent. The substantially transparent layer 408 may be provided in the form of a glass lid that hermetically seals the sensor die 416 inside the cavity of the body 404. The externally exposed surface of the substantially transparent layer 408 is shown to be sufficiently flat or planar so as to facilitate contact between the analyte sensing device 128 and the one or more analytes. The chemochromic layer 412 is conformal to the underlying externally exposed surface of the substantially transparent layer 408. In some embodiments, the chemochromic layer 412 may be coaxially aligned with the perimeters of the substantially transparent layer 408, meaning that the chemochromic layer 412 substantially covers the entire top surface of the substantially transparent layer 408.

The analyte sensing device 128 is also shown to include an optical element 420. The optical element 420 is shown as a lens (e.g., having a non-planar surface) that may help to focus light on photosensitive areas of the sensor die 416. The optical element 420 may be provided as a transparent (fully or partially) epoxy or encapsulant (e.g., silicone) that also helps to seal and protect the sensor die 416 within the cavity of the body 404. It should be appreciated that the optical element 420 is an optional component, but may be useful to increase the amount or quality of light that is received at the sensor die 416.

In one embodiment, the chemochromic layer 412 fully extends over the externally exposed surface of the substantially transparent layer 408. Such a configuration may be suitable for detecting a single analyte, or a limited set of analytes which have limited or predetermined manner of color changes such that the color changes can be detected using a set of color sensors provided on the sensor die 416.

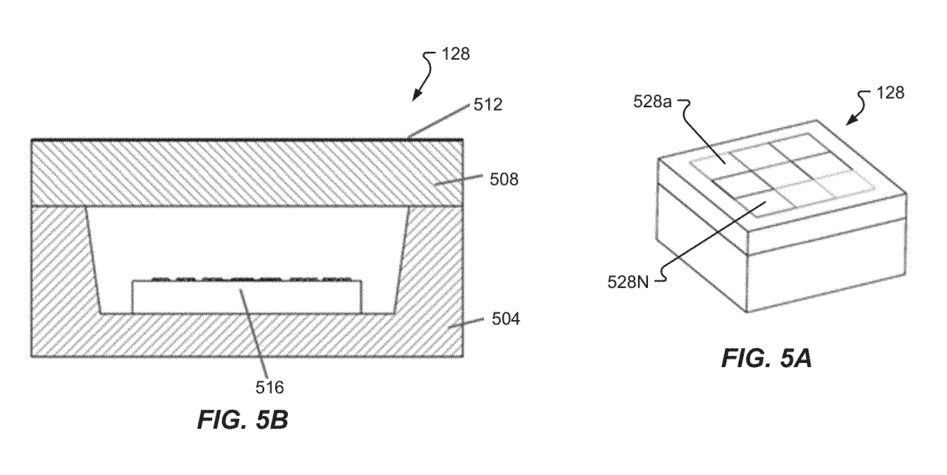

In some embodiments, the chemochromic layer may comprise a plurality of chemochromic materials. An example of such a configuration will now be described with reference to FIGS. 5A-B. For example, the chemochromic layer 512 may have N different chemochromic materials 528a-N arranged in an array or other two dimensional manner (e.g., a row or columnar format). Each of the chemochromic materials 528a-N may be selected to be responsive to a predetermined analyte or set of analytes. For example, the first chemochromic material 528a may change to color P if exposed to analyte X, but may change to color Q if exposed to analyte Y. Another chemochromic material 528N may change to different colors in response to exposure to other analytes.

To detect any color change, the sensor die 516 is provided with at least three detectors for each analyte. By way of example, detectors or sensors such as RGB sensor, or CMY sensors may be used with the sensor die 516. Other suitable sensors which could detect the change in color of the chemochromic layer may also be useful. To have higher precision, the sensor die 516 may have at least four detectors for each analyte 528a-N, for example, a RGB sensor and a clear photo-sensor. However, as each analyte 528a-N is configured to change color in a limited manner, the sensor die 516 may not need three or four detectors for each analyte. In some cases, a set of two detectors may be sufficient to detect color change of the chemochromic materials 528a-N. When there is more than one chemochromic material in the chemochromic layer 512, the sensor die may 516 comprise sets of detectors arranged approximating the chemochromic materials 528a-N.

The analyte sensing device 128 of FIGS. 5A-B is otherwise similar to the analyte sensing device of FIGS. 4A-B in that the sensing die 516 is provided in a cavity of the body 504 and the substantially transparent layer 508 is provided as a lid for the body 504. The analyte sensing device 128 of FIGS. 5A-B, however, is not shown to include an optical element. It should be appreciated that the analyte sensing device 128 for sensing multiple analytes may be provided with an optical element without departing from the scope of the present disclosure.

Another consideration for designing the number of detectors is the alignment of the chemochromic materials relative to the detectors of the sensor die. Generally, the sensor die is placed at a distance approximately more than ten times the detector size. Each detector may have a size or sensing area of a few microns. Therefore, alignment of the detectors to the chemochromic materials may not be ideal.

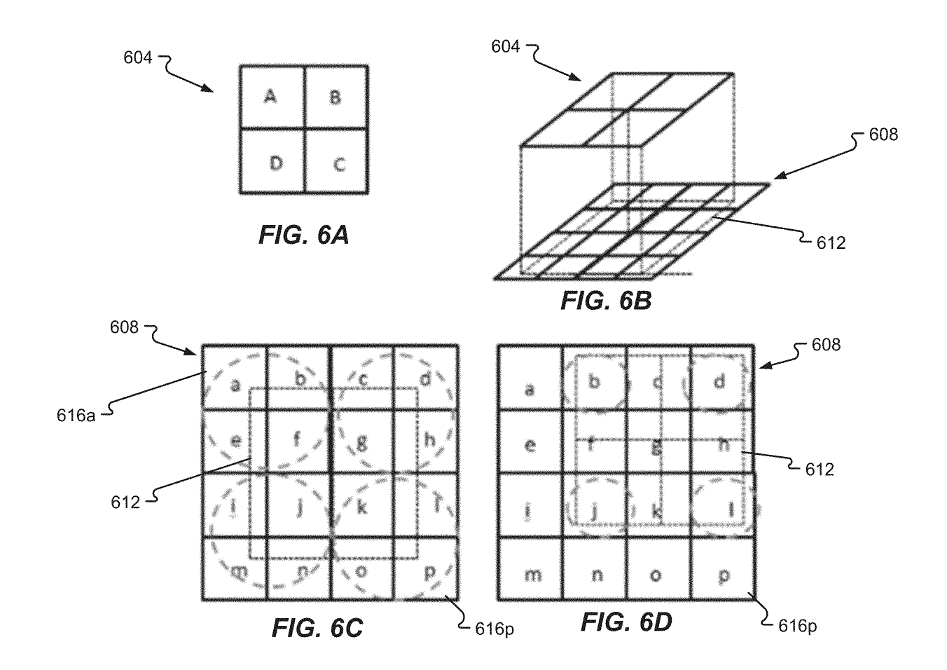

FIGS. 6A-D provide illustrative diagrams showing the design considerations of the chemochromic materials relative to the plurality of detectors. For example, FIG. 6A shows a chemochromic layer 604 having a plurality of different chemochromic materials. The illustrative chemochromic layer 604 of the analyte sensing device 128 is shown to include four chemochromic materials arranged in an array (e.g., chemochromic material A, chemochromic material B, chemochromic material C, and chemochromic material D). It should be appreciated that a greater or lesser number of chemochromic materials may be included in the chemochromic layer 604 without departing from the scope of the present disclosure. It should also be appreciated that the chemochromic layer 604 may be provided in any of the analyte sensing devices 128 depicted and described herein. Each of the chemochromic materials in the chemochromic layer may respond to a set of analytes, which may or may not be the same set of analytes.

Generally, the set of analytes detectable by one chemochromic material are selected such that the chemochromic material responds differently to each analyte. For example, analyte A and analyte B both result in a chemochromic material changing from transparent to a red color. In this example, it is preferable to have the chemochromic material A configured to detect analyte A and have a different chemochromic material B to detect analyte B. If chemochromic material A is configured to have a color change to red in respond to both analyte A and analyte B, detection of color change may not be able to determine presence of analyte A, or analyte B. However, the chemochromic material A may be selected to detect analyte C (which resulted in color change to blue), and analyte D (which resulted in color change to green).

Each set of detectors 608 provided on a sensor die may comprise a RGB sensor, a CMY sensor, a RGB and clear photodiode sensor, a RGB and covered photodiode sensor, a combination of interference filter or any combination thereof in order to detect color changes of a chemochromic material in the chemochromic layer 604. The set of detectors 608 may be distributed across the detection surface of the sensor die. As the changes of color in each chemochromic material is a predetermined known set of choices, the number of sensors in each detector may be further optimized or reduced. In one embodiment, the sensor die may comprise two color sensors.

As shown in FIG. 6B, the sensor die may comprise a set of detectors 608 arranged at a distance away from the chemochromic layer 604. The chemochromic layer 604 is externally exposed on the substantially transparent layer. On the other hand, the sensor die is generally concealed on an opposite side of the substantially transparent layer (e.g., sealed and protected by the substantially transparent layer). In FIG. 6B, each set of detectors 608 is represented by one of the squares in the array. For example, the set of detectors 608 may comprise two or more detectors therein. In other words, the sensor die may comprise a plurality of sets of detectors arranged in an array as shown in FIGS. 6C and/or 6D. Each set of detectors may comprise equal number of detectors. Each detector in the same set may have different wavelength characteristic. Each set of detectors may have similar composition of detectors. For example, a plurality of detectors 616a-p may be provided in the set of detectors 608 and the plurality of detectors 616a-p may be provided in an array configuration. The chemochromic layer 604 may be positioned over the set of detectors 608 such that there is an overlap area 612 between the chemochromic layer 604 and set of detectors 608 that intersects each of the plurality of detectors 616a-p. In some embodiments, the center of the chemochromic layer 604 may substantially align with the center of the set of detectors 608, in which case the overlap area 612 completely covers the center detectors 616f, 616g, 616j, and 616k as shown in FIG. 6C. Alternatively, because the set of detectors 608 is larger in area than the overlap area 612, off-axis alignments may be accommodated as shown in FIG. 6D. This may enable machining and manufacturing tolerances to be accommodated. In some embodiments, the overlap area 612 may actually correspond to an illumination area, which may not necessarily match the size of the chemochromic layer 604 due to an optical element being positioned between the chemochromic layer 604 and set of detectors 608. If an optical element is used, then the size of the area illuminated at the set of detectors 608 may be larger or smaller than the size of the area covered by the chemochromic layer 604. In some embodiments, each of the detectors 616a-p may include a plurality of detectors (e.g., each detectors 616a-p may have a red detector, a blue detector, and a green detector).

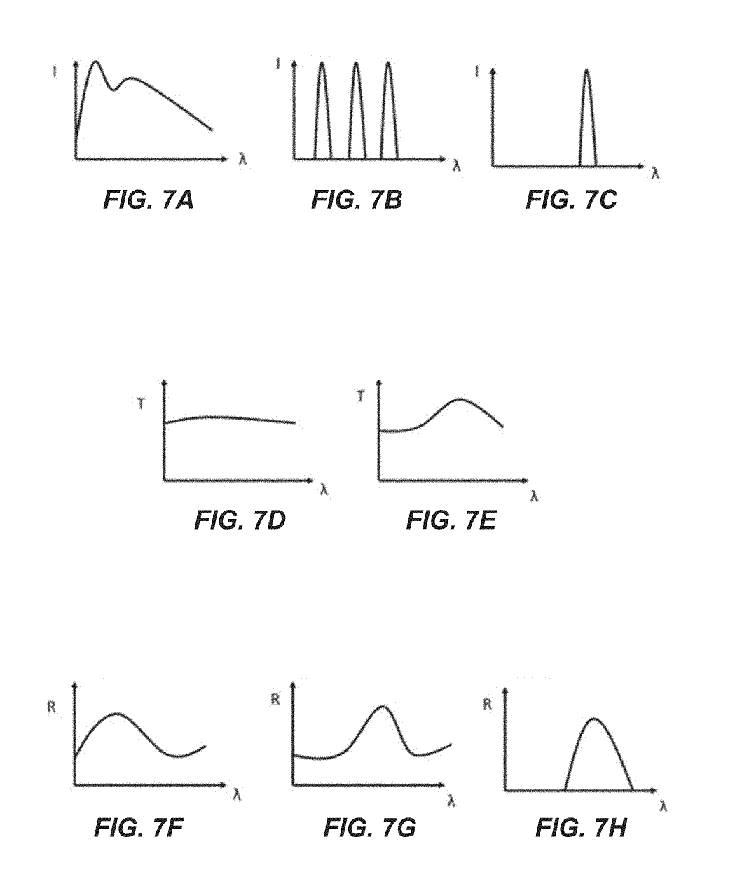

The output of the detectors 616a-p is a factor of the spectral profile of the illumination source (including external radiation 240 or internal radiation 236), the spectral response of the chemochromic materials in each state, and the spectral response of the detectors. FIGS. 7A-H depict various examples of such outputs.

FIGS. 7A-C show three spectral profiles of three different illumination source. The X-axis represents the wavelength whereas the Y-axis represents intensity of light detected at each wavelength. For example, if the sensing device is illuminated by light sources in a room using a white LED, the spectral profile may be similar to the profile shown in FIG. 7A. In comparison, FIG. 7B shows a spectral profile of a RGB LED light source. As yet another example, FIG. 7C shows a spectral profile of a single-wavelength light source (e.g., a red light source or red LED).

FIGS. 7D-E depict examples of transmission profiles of a chemochromic material in a first state and in a second state. The X-axis represents the wavelength whereas the Y-axis represents the transmissivity of the chemochromic layer (e.g., the amount of light being passed through the chemochromic layer). In the first state, as shown in FIG. 7D, the chemochromic material is sufficiently thin to allow a majority of light to pass through regardless of wavelength. For example, the chemochromic layer is sufficiently thin to allow at least 30% of an external radiation to pass there through. After being in contact with a predetermined analyte, the chemochromic material may change color, for example to red, which has a profile as shown in FIG. 7E. The peak of the transmission profile is at around 630nm, and therefore, the chemochromic material may appear red.

FIGS. 7F-H show three examples of a spectral response of three different detectors. The X-axis represents the wavelength whereas the Y-axis represents output of the detectors at each wavelength. The detectors, for example, may be coated with a color filter or an interference filter. FIG. 7F corresponds to an output of a photosensor/detector coated with a blue pigment color filter. FIG. 7G corresponds to an output of a photosensor/detector coated with a red pigment color filter. The organic-based pigment color filter may have a profile allowing a small portion of light at other wavelength to pass through. For example, the blue pigment color filter may allow some components of red wavelength to pass through. FIG. 7H corresponds to an output of a photosensor/detector coated with an interference filter (reflective or absorptive). The interference filter may be designed to reject any wavelength (e.g., a predetermined and selected wavelength).

Each of the detectors may be configured to detect radiation having different wavelength characteristics. For example, the detector in FIG. 7F may be primarily used to detect blue light. To detect red light, one of the detectors in FIG. 7G or 7H may be employed. However, the output of a single detector may not be able to differentiate a situation where the color change is caused by the illumination source. For example, consider Scenario A where an illumination source changes from a white LED to red LED or changes in response to a change in the chemochromic material. Consider also Scenario B in which the chemochromic material exhibits a color change due to exposure to the analyte. In both Scenario A and Scenario B, the detector having the profile shown in FIGS. 7G or 7H, which is mainly used to detect red light (e.g., a radiation having a wavelength characteristic which peaks at primary red wavelength) may both exhibit an increased output, thereby rendering it difficult to distinguish the source of color change. However, by using two detectors, the source of color change may be determined. In the example illustrated above, the detector having a profile as shown in FIG. 7F would have a higher output when the changes of color occurs at the chemochromic layer (e.g., Scenario B) as compared to a situation where the output caused by changes of the illumination source (e.g., Scenario A) because the illumination source of red LED may have zero or substantially negligible components of blue wavelength.

Above are simple examples for illustrative purposes and may not reflect an actual design. The determination of color may be more complicated involving careful calibration and use of software to carry out a much more complicated algorithm to determine source of a color change. In addition, the determination of color may be carried out using detectors which detect light from the illumination source directly without passing through the chemochromic material as shown in next few paragraphs. Alternatively or in addition to the above, the detector may be configured to compare an output of an earlier time period to determine color change at a particular point in time.

The sensor die may have more detectors than the number of chemochromic materials. The detectors may be connected to a switching circuit and a control circuit (e.g., provided in the form of the microprocessor 108) so as to determine the color change in each of the chemochromic materials. For example, for four chemochromic materials shown, the sensor die may have 16×3 detectors. A greater number of detectors may enable detection of color without proper alignment between the chemochromic layer and the sensor die as shown in FIG. 6D. For example, when the chemochromic layer and the sensor die are aligned in an ideal manner as shown in FIG. 6C, detectors (or detector sets) 616a, 616b, 616e, and 616f will be producing an output corresponding to the chemochromic material A. Detectors (or detector sets) 616c, 616d, 616g, and 616h will be producing an output corresponding to the chemochromic material B. Detectors (or detector sets) 616i, 616j, 616m, and 616n will be producing an output corresponding to the chemochromic material C. Detectors (or detector sets) 616k, 616l, 616o, and 616p will be producing an output corresponding to the chemochromic material D. In addition, the detector 616f will be producing an output almost 100% corresponding to the changes of the chemochromic material A, whereas detector 616a may not be as responsive as the detector 616f because the detector 616a may be exposed to illumination directly without passing through the chemochromic material A.

Throughout the manufacturing process, it may be desirable not to allow the chemochromic materials to go through a color change. For calibration purposes, one or more alignments marks may be placed adjacent to the chemochromic materials. For example, the boundary (e.g., outer edge, a particular corner, or all outer edges) of the chemochromic materials may have alignments marks provided thereon.

The switching circuit and the control circuit (e.g., in the microprocessor 108) may be configured to compare output of the detectors, for example the detector 616a and the detector 616f to determine whether the changes of output detected is caused by the changes of color in light source (e.g., emitter 228 or ambient light 240), or by the changes of color in the chemochromic material A. If the color change happens at the illumination source, both detector 616a and 616f may observe similar changes. However, if the color change takes place at the chemochromic material A, detector 616f may observe more changes in output as compared to the detector 616a. Another way to determine the source of change is by monitoring how fast the color change takes place. This may be detected by employing the timer circuitry 140.

In most circumstances, as placing of components is done generally with an accuracy of 5 microns to 50 microns, the alignment should not be assumed to be ideal. The example shown in FIG. 6D highlights that detectors (or detector sets) 616b, 616d, 616j, and 616l may be primarily used to detect changes of colors in chemochromic materials A, B, C, D, respectively. Changes of output in other detectors may be due to the illumination source, or a combination of effects due to multiple chemochromic materials. Calibration may be carried out and each detector 616a-p may be analyzed using a software run on external computers or microprocessors 108. For this purpose, the control circuit may have a communication port configured to establish a communication between the control circuit and the external processor. In some embodiments, the communication port may be a serial communication port such as an I2C communication port. The switching circuit and the control circuit may be external circuits coupled to the sensor die. Alternatively, the control circuit and the switching circuit may be part of the sensor die.

The analyte sensing device 128, for example, may optionally include an optical element. The optical element, for example, may include a lens structure. The optical element may be formed within the substantially transparent layer, or alternatively, the optical element may be formed as a separate structure within the cavity as shown in FIG. 4B. The optical element is configured to direct radiation to the detectors or detector sets. The optical element, in one embodiment, substantially covers the detectors of the sensor die.

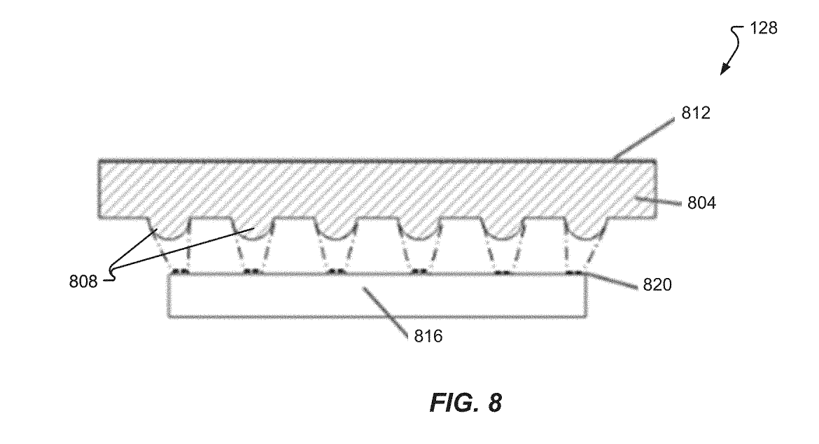

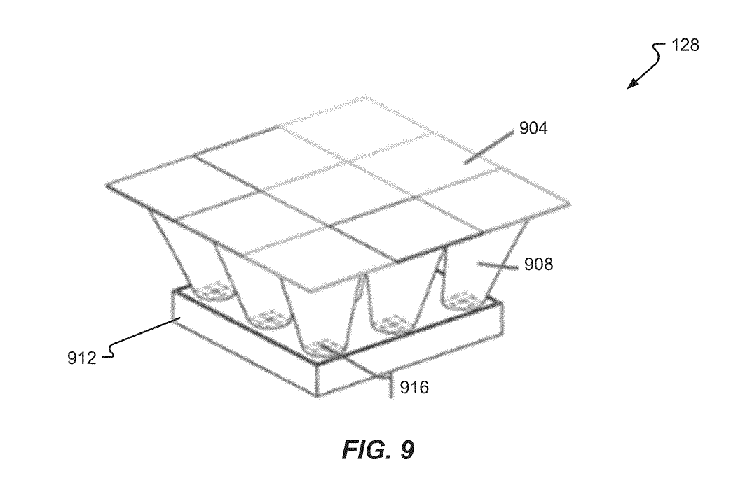

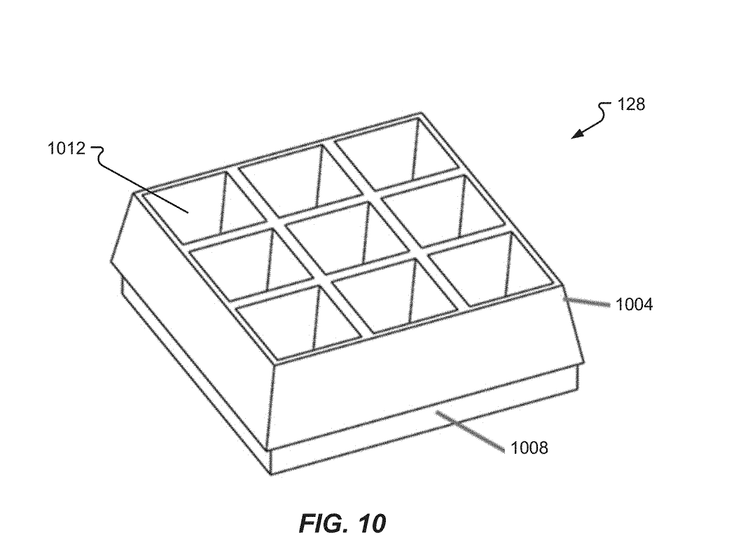

Alternatively, the optical element is disposed on an internal surface of the substantially transparent layer as illustrated in FIG. 8. More specifically, the analyte sensing device 128 may include a substantially transparent layer 804 having a chemochromic layer 812 on one side (e.g., the externally exposed side of the substantially transparent layer 804) and one or more lenses 808 formed on its opposing side (e.g., the internal surface of the substantially transparent layer 804). This internal surface of the substantially transparent layer 804 may face the sensor die 816 and the detector areas 820 provided thereon. As can be seen in FIG. 8, the optical elements 808 disposed on the internal surface of the substantially transparent layer 804 may be provided in the form of one or many microlenses. Other suitable lens configurations may also be utilized without departing from the scope of the present disclosure. Each optical element may be useful to focus light passing through the substantially transparent layer 804 (or reflecting off the top surface of the substantially transparent layer 804) onto the detector area(s) 820 of the sensor die 816.

As described above, in some embodiments, the sensor die includes a set or sets of detectors and the chemochromic layer includes a plurality of chemochromic materials. In such a configuration, the analyte sensing device 128 may comprise a plurality of optical elements that are arranged such that each of the plurality of chemochromic portions is optically coupled to a predetermined set of detectors through one or more of the plurality of optical elements. The plurality of optical elements may be provided in the form of lens 808. Alternatively or additionally, one or more of the optical elements that optically couple the sensor die with the substantially transparent layer may be provided in the form of a light guide. An example of such a configuration is shown in FIG. 9.

The analyte sensing device 128 of FIG. 9 is shown to include a sensor die 912 with a plurality of detectors 916 provided thereon. The sensor die 912 receives light that passes through the chemochromic layer 904. In this particular embodiment, the optical elements 908 positioned between the chemochromic layer 904 and the sensor die 912 is in the form of one or many light guides. As shown, the light guides 908 are positioned between one of the plurality of detectors 916 and one of the plurality of chemochromic portions. The light guide 908, as shown, establishes an optical communication channel between the detector 916 and the chemochromic layer 904.

In some embodiments, it may be desirable to maintain optical isolation between the detectors or detector areas. FIG. 10 depicts one example of an analyte sensing device 128 that enables such optical isolation. The analyte sensing device 128 is shown to include a sensor die 1008 with an optical isolation element 1004 provided thereon. The optical isolation element 1004, for example, includes individual compartments 1012 that optically isolate each detector on the sensor die 1008. Each of the compartments 1012 is positioned adjacent to one of the plurality of detectors and one of the plurality of chemochromic portions so as to define an optical communication channel therebetween. The optical isolation element 1004 may be sandwiched between and in direct physical contact with the sensor die 1008 and the substantially transparent layer. The substantially transparent layer is not depicted in FIG. 10 so as not to obscure the depiction of the optical isolation element 1004.

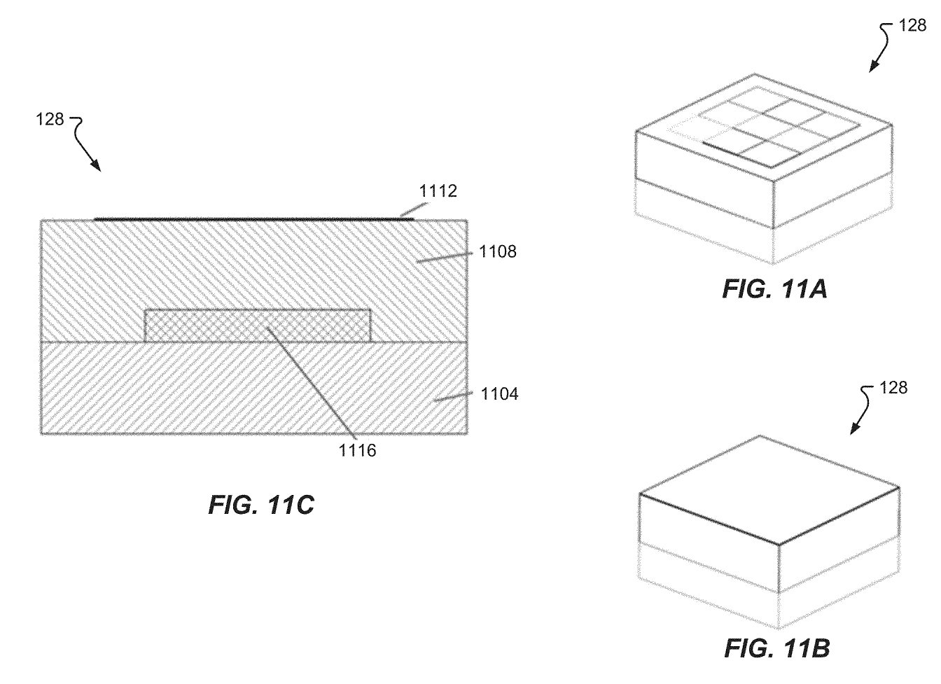

As described above, the body of the analyte sensing device 128 may be fashioned to include a cavity and the sensor die may be disposed within the cavity of the body. In another embodiment, the body does not necessarily need to include a cavity. An example of such a configuration for the analyte sensing device 128 is shown in FIGS. 11A-C. It should be appreciated that such a configuration may be used for an analyte sensing device used to detect multiple analytes (e.g., FIG. 11A) or a single analyte (e.g., FIG. 11B). In some embodiments, the analyte sensing device 128 includes a sensor die 1116 mounted on a body 1104. The body 1104, for example, may be a PCB substrate. Other suitable substrates may also be used for the body 1104. As shown, the sensor die 1116 is disposed on the receiving surface of the body 1104 and the substantially transparent layer 1108 comprises a clear molding material configured to encapsulate the sensor die 1116.

The externally exposed surface of the substantially transparent material 1108 is shown to have the chemochromic layer 1112 provided thereon. The chemochromic layer 1112, in this example, may correspond to a chemochromic material that is deposited on the substantially transparent material 1108 after the substantially transparent material 1108 has been formed and cured around the sensor die 1116. Alternatively, the chemochromic material may be integrated into the material of the substantially transparent material 1108 (as shown in FIG. 3C), in which case the sensor die 1116 is surround on its top and sides by the chemochromic material.

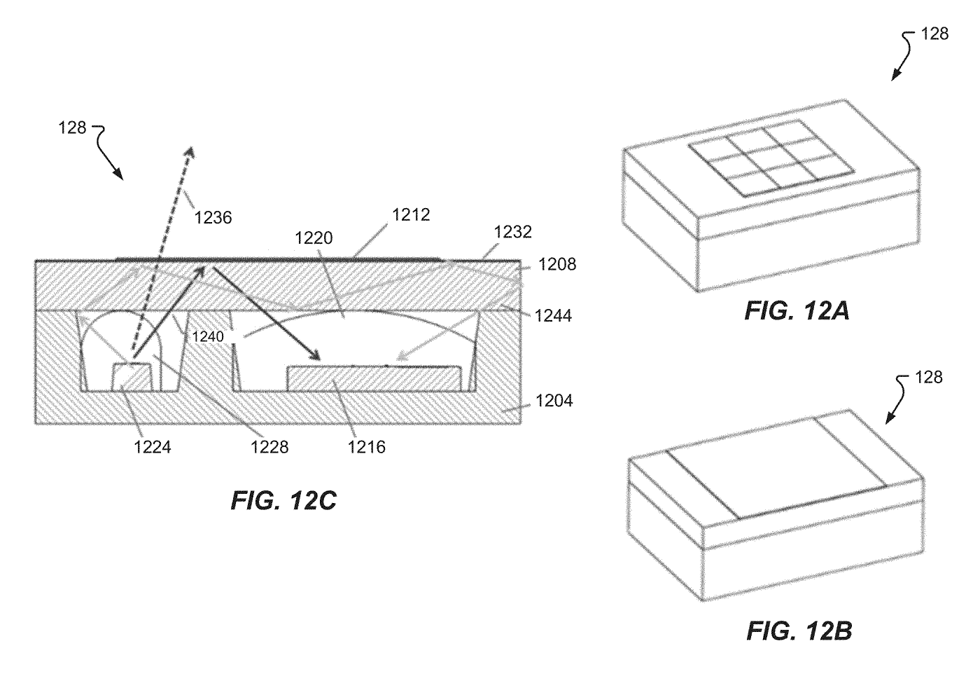

In some applications, such as when the analyte sensing device 128 is provided in a wearable device, an external radiation or illumination source may not be readily available to illuminate the chemochromic material. FIGS. 12A-C depict an alternate design of an analyte sensing device 128 which may be useful for such applications. As shown, the analyte sensing device 128 may further comprise an emitter die 1224 in addition to the sensor die 1216. The emitter die 1224 may operate as a light source or a radiation source configured to emit a radiation 1236, 1240 towards the chemochromic layer 1212. The radiation may be visible light or invisible light such as an ultra violet or infrared. In one embodiment, the emitter die 1224 may be a LED.

The analyte sensing device 128 is shown to include an emitting optical element 1228 which is in optical communication with the emitter die 1224 and a receiving optical element 1220 which is in optical communication with the sensor die 1216. As shown, the emitting optical element 1228 and the receiving optical element 1220 may encapsulate the emitter die 1224 and the sensor die 1216, respectively. The optical elements 1220, 1228, as shown, for example, may be optical lenses attached to the substrate or body 1204 of the analyte sensing device 128. The emitting optical element 1228, for example, is configured to direct a radiation 1236, 1240 from the emitter die 1224 to the chemochromic layer 1212 such that a substantial portion of the radiation 1240 is reflected towards the sensor die 1216.

In one embodiment, the body includes a first cavity and a second cavity. As shown, the first cavity is isolated from the second cavity through a portion of the body. The first cavity and the second cavity may be configured to accommodate the emitting optical element and the receiving optical element respectively such that the radiation emitted by the emitter die may be transmitted through the first cavity towards the chemochromic layer and reflected off the chemochromic layer towards the sensor die.

In one embodiment, the substantially transparent layer 1208 is provided with the chemochromic layer 1212 thereon and is configured to direct the reflected radiation 1240 towards the sensor die 1216. A surface treatment 1232 may be provided on the externally exposed surface of the substantially transparent layer 1208 to direct a majority of the reflected radiation 1240, 1244 towards the sensor die 1216. In one example, the substantially transparent layer 1208 comprises a microlens that is configured to direct the reflected radiation 1240, 1244 towards the sensor die 1216. The microlens, for example, is disposed within the substantially transparent layer 1208 such that the reflected radiation 1240, 1244 is guided towards the sensor die. Further, the emitter die 1224, the emitting optical element 1228, the substantially transparent layer 1208 and the sensor die 1216 may be arranged to achieve a total internal reflection such that less than 50% of the radiation emitted from the emitter die exits through the externally exposed surface (e.g., in the form of radiation 1236).

In addition, instead of providing the emitting optical element 1228 in the form of a lens, a reflector structure may be provided over the emitter die 1224 as an alternative form of an optical element. The reflector, in one embodiment, is configured to direct the radiation from the emitter such that a substantial portion of the radiation is reflected towards the sensor die 1216.

The package body 1204 may be similar to a conventional proximity sensor. However, unlike the conventional proximity sensor which requires the light to be emitted towards an object further away as illustrated by radiation 1236, the package body 1204 for the analyte sensing device 128 is designed to illuminate the chemochromic layer 1212 so as to be detected by the sensor die 1216 as illustrated by radiation 1240. In another embodiment, the sidewalls of the reflector may be adjusted to different angles and thus may not be symmetrical as shown in FIG. 12C. Furthermore, the substantially transparent layer 1208 may act as a light guide (with the assistance of the microlens) for directing light from the emitter die 1224 towards the sensor die 1216 as illustrated by radiation 1244.

In some embodiments, chemochromic materials provided in the chemochromic layer 1212 may require active resetting to its original or first state so that it can be reused within a short time frame. In such case, the emitter die 1224 may be configured to emit a radiation towards the chemochromic layer 1212 so as to change the second color of the chemochromic material to the first color. Alternatively, an additional emitter for resetting the chemochromic layer 1212 may be added in addition to the emitter die 1224.

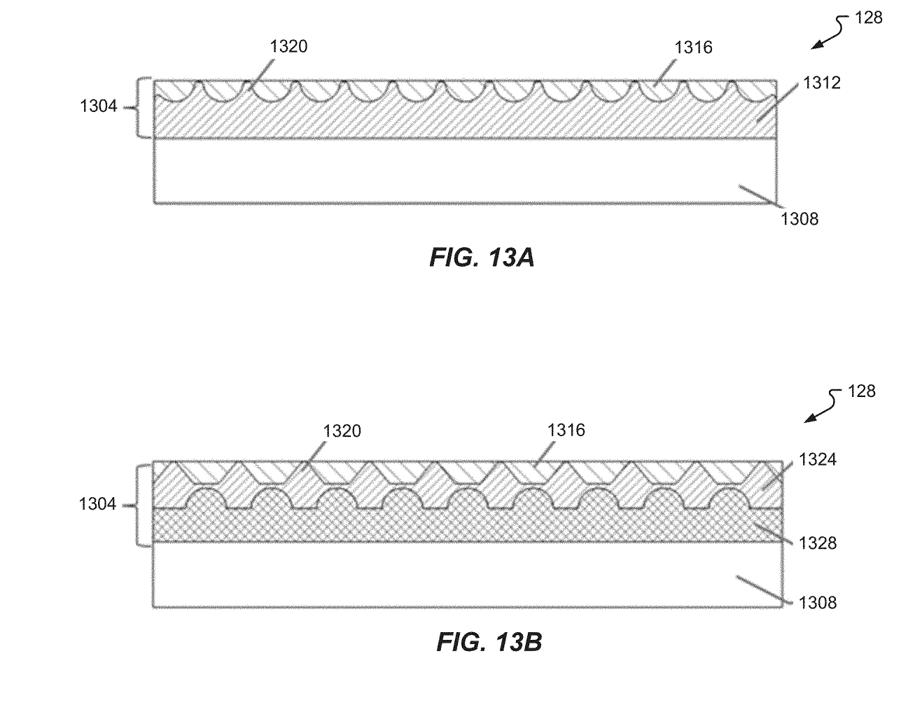

With reference now to FIGS. 13A-B, alternative designs of an analyte sensing device 128 for detecting one or more analytes will be described in accordance with at least some embodiments of the present disclosure. Such designs improve durability of the analyte sensing device 128. In one embodiment, the substantially transparent layer 1304 comprises an interlocking structure 1320 so as to establish a mechanical interlock between the chemochromic layer 1316 and a clear mold 1312 of the substantially transparent layer 1304. The interlocking structure 1320, for example, comprises a plurality of mesas defining the chemochromic layer 1316 into a plurality of wells of chemochromic materials. In one embodiment, the plurality of mesas define each of the plurality of wells of chemochromic materials into a lens shaped structure for directing radiation in a predetermined direction.

In another embodiment as shown in FIG. 13B, the substantially transparent layer 1304 comprises a first encapsulant layer 1324 having a first reflective index and a second encapsulant layer 1328 having a second reflective index that is different than the first reflective index. The first encapsulant layer 1324 is sandwiched between the chemochromic layer 1316 and the second encapsulant layer 1328. The interlocking structure 1320, for example, is formed on the first encapsulant layer 1324, while the second encapsulant layer 1328 comprises a plurality of lenses adjacent to the plurality of wells of chemochromic materials.

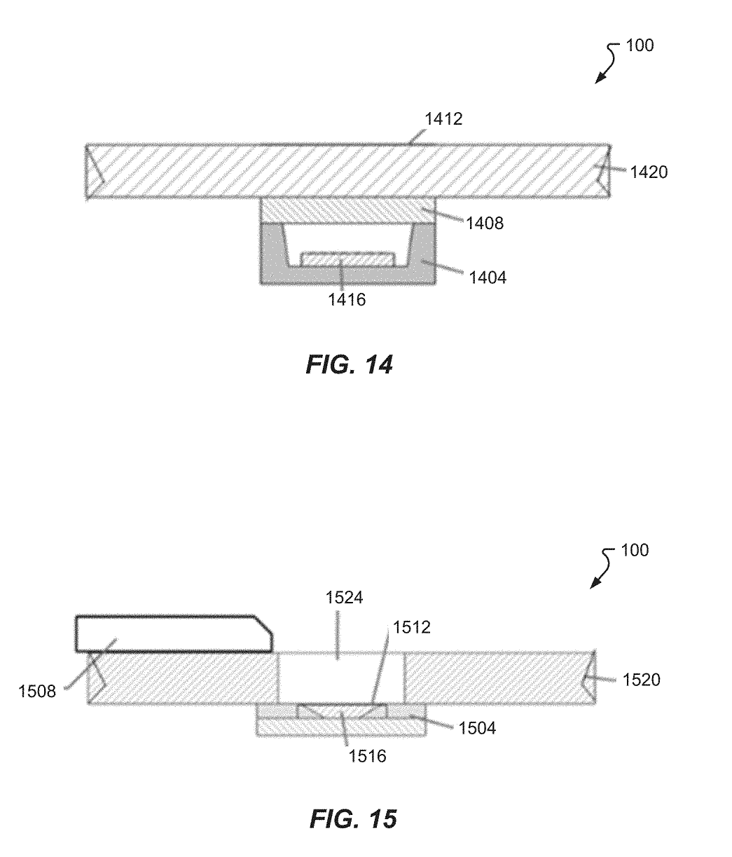

With reference now to FIGS. 14 and 15, details of different ways to incorporate an analyte sensing device 128 into a mobile device 100 will be described in accordance with at least some embodiments of the present disclosure. FIG. 14 depicts a portion of a mobile device 100 having the analyte sensing device 128. In the depicted embodiment, the analyte sensing device 128 forms a portion of a portable device having a casing or housing, which may also be referred to as a package body 1404. The package body 1404 comprises a cavity to receive the sensor die 1416—similar to the embodiments depicted in FIGS. 4A-B. The package body 1404 is covered with the substantially transparent layer 1408, which may be provided as a lid to cover the cavity of the package body 1404. In the depicted embodiment, the chemochromic layer 1412 is disposed on a housing 1420 of the mobile device 100. In other words, the substantially transparent layer 1408 is a part of the casing or housing 1420 of the mobile device 100 in this embodiment.

In contrast to the configuration depicted in FIG. 14, the mobile device 100 may alternatively be provided with a housing 1520 having an opening 1524 provided therein. The opening 1524 in the housing 1520 is provided to accommodate the analyte sensing device 128 and to expose the chemochromic layer 1512 of the analyte sensing device 128 to an external environment (and therefore an analyte).

An optional movable protector 1508 may be provided on the housing 1520 of the mobile device 100, thereby enabling an optional exposure and covering of the opening 1524. The movable protector 1508 covers the opening in a first position (not shown) and the movable protector exposes the opening in a second position as illustrated.

This particular embodiment also shows a package body 1504 having a sensor die 1516 mounted thereon, but the package body 1504 is in direct contact with the housing 1520 of the mobile device 100. Such a configuration allows the chemochromic layer 1512 of the sensing device 128 to be in fluid communication with the one or more analytes that pass through the opening 1524. In some embodiments, the externally exposed surface of the substantially transparent layer and a surface of housing 1520 are substantially coplanar with each other, meaning that the analyte sensing device 128 is mounted flush with respect to the outer surface of the housing 1520.

In some embodiments, the chemochromic layer 1512 exhibits a first color in a first state, and a second color in a second state when exposed to a predetermined analyte. The sensor die 1516 is configured to detect the change in color of the chemochromic layer 1512. The sensor die 1516, as shown, is housed in a package body 1504. The body forms a cavity such that the cavity is approximating the opening of the housing. The chemochromic layer is disposed within the cavity such that the opening, the cavity and the chemochromic layer are in fluid communication with each other.

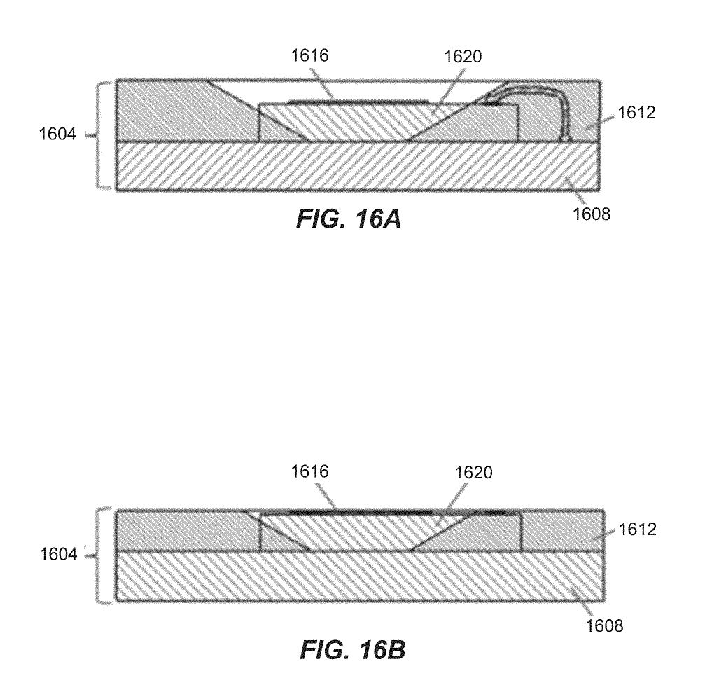

With reference now to FIGS. 16A-B, various package configurations suitable for an analyte sensing device 128 will be described in accordance with at least some embodiments of the present disclosure. FIG. 16A depicts first configuration in which one or more wire bonds are used to electrically connect the sensor die 1620 to one or more electronic traces or contacts on the substrate 1608. In this embodiment, the chemochromic layer 1616 is disposed directly on the sensor die 1620 instead of a substantially transparent layer. The sensor die 1620 comprises a passivation layer on its top surface to accommodate the application of the chemochromic layer 1616 thereon. Thus, the chemochromic layer 1616 is disposed directly on the passivation layer.

In some embodiments, the at least one wire bond is encapsulated within the package body 1604. The package body 1604 comprises a substrate 1608 and an upper portion 1612 positioned adjacent to the substrate 1608. The upper portion 1612 of the body 1604 has an opening adjacent to the chemochromic layer 1616.

FIG. 16B depicts an alternative arrangement whereby the sensor die 1620 is flip-chip bonded to the substrate 1608. Thus, one or more solder bumps may be used to connect electrical connectors or bonding pads on the sensor die 1620 to corresponding bonding pads on the substrate 1608. This particular type of configuration may enable a thinner package body 1604 vis-à-vis a thinner upper portion 1616.

Specific details were given in the description to provide a thorough understanding of the embodiments. However, it will be understood by one of ordinary skill in the art that the embodiments may be practiced without these specific details. In other instances, well-known circuits, processes, algorithms, structures, and techniques may be shown without unnecessary detail in order to avoid obscuring example embodiments.

While illustrative embodiments have been described in detail herein, it is to be understood that inventive concepts may be otherwise variously embodied and employed, and that the appended claims are intended to be construed to include such variations, except as limited by the prior art.

What is claimed is:

1. A sensing device for detecting one or more analytes, the sensing device comprising:
a body;
a sensor die; and
a substantially transparent material positioned such that the sensor die is sandwiched between the body and the substantially transparent material, wherein the sensor die is in optical communication with the substantially transparent material and in electrical communication with the body, and wherein the substantially transparent material comprises:
a detection surface exposed such that the detection surface is adaptable to be in direct contact with the one or more analytes; and
a chemochromic material disposed at least partially adjacent to the detection surface such that a portion of the chemochromic material is configured to be exposed to the one or more analytes through the detection surface, wherein
the chemochromic material exhibits a first color in a first state, and a second color in a second state when exposed to a predetermined analyte,
the sensor die is configured to detect the change in color of the chemochromic material, and
the chemochromic material, the detection surface, and the substantially transparent material are integrally formed in a semiconductor package.

2. The sensing device of claim 1 further comprising:
an emitter arranged such that an optical signal emitted from the emitter is directed to the substantially transparent material so as to be reflected to the sensor die by the detection surface after passing through the chemochromic material.

3. The sensing device of claim 1, wherein the chemochromic material is sufficiently thin such that at least 30% of external radiation passes through the chemochromic material in the first state or the second state.

4. The sensing device of claim 1, wherein the chemochromic material fully extends over the exposed surface.

5. The sensing device of claim 1, wherein the sensor die is configured to produce an output which corresponds to a spectral profile of an external radiation passing through the chemochromic material and a spectral response of the chemochromic material.

6. The sensing device of claim 1, wherein the change of color in the chemochromic material is within a first time period and wherein the sensing device further comprises a timer circuitry to determine a length of the first time period.

7. The sensing device of claim 1 further comprising an optical element configured to direct the radiation to each of the at least two detectors.

8. The sensing device of claim 7, wherein the optical element is disposed on an internal surface of the substantially transparent layer, wherein the internal surface opposes the exposed surface.

9. The mobile device of claim 1, wherein the substantially transparent layer comprises an alignment mark adjacent to at least one of a plurality of chemochromic portions.

10. The sensing device of claim 9 further comprising a plurality of optical elements and a plurality of detectors, wherein each of the plurality of chemochromic portions is optically coupled to a predetermined set of detectors through one or more of the plurality of optical elements.

11. The sensing device of claim 10, wherein each of the plurality of optical elements comprises an optical isolation element positioned adjacent to one of the plurality of detectors and one of the plurality of chemochromic portions so as to define an optical communication channel therebetween.

12. The sensing device of claim 10 further comprising:
a circuit configured to electrically connect a predetermined set of the plurality of detectors such that the predetermined set of the plurality of detectors provide an output that corresponds with one of the plurality of chemochromic portions.

13. The sensing device of claim 1, wherein the sensing device forms a portion of a portable device having a casing, wherein the substantially transparent layer is a part of the casing.

14. The sensing device of claim 1 further comprising:
an emitter die configured to illuminate the chemochromic material.

15. The sensing device of claim 2 further comprising:
an emitting optical element configured to direct a radiation from the emitter to the chemochromic material such that a substantial portion of the radiation is reflected towards the sensor die.

16. The sensing device of claim 15, wherein the emitter, the emitting optical element, the substantially transparent layer, and the sensor die are arranged to achieve a total internal reflection such that less than 50% of the radiation emitted from the emitter exits through the detection surface.

17. The sensing device of claim 2, further comprising:
a reflector configured to direct a radiation from the emitter to the chemochromic material such that a substantial portion of the radiation is reflected towards the sensor die.

18. The sensing device of claim 1 wherein the substantially transparent layer comprises an interlocking structure that establishes a mechanical interlock between the chemochromic material and the substantially transparent layer.

19. The sensing device of claim 18, wherein the interlocking structure comprises a plurality of mesas defining the chemochromic material into a plurality of chemochromic wells.

20. The sensing device of claim 18, wherein the substantially transparent layer comprises a first encapsulant layer having a first reflective index and a second encapsulant layer having a second reflective index that is different than the first reflective index, and wherein the interlocking structure is formed on the first encapsulant layer.

21. A mobile device comprising:
a housing having an opening;
a sensor die disposed within the housing adjacent to the opening, wherein the sensor die comprises:
a detection surface exposed externally such that the detection surface is adaptable to be in direct contact with one or more analytes, and
a chemochromic material disposed at least partially adjacent to the detection surface such that a portion of the chemochromic material is configured to be exposed through the detection surface, wherein
the chemochromic material exhibits a first color in a first state, and a second color in a second state when exposed to a predetermined analyte,
the sensor die is configured to detect the change in color of the chemochromic material, and
the chemochromic material, the detection surface, and the sensor die are integrally formed in a semiconductor package.

22. The mobile device of claim 21, wherein the sensor die comprises a passivation layer on a top surface, and wherein the chemochromic material is provided in a chemochromic layer that is disposed on the passivation layer.

23. The mobile device of claim 22, wherein:
the semiconductor package comprises a body; and
the body forms a cavity such that the cavity approximates the opening of the housing.

24. The mobile device of claim 23, wherein chemochromic layer is disposed within the cavity such that the opening, the cavity, and the chemochromic layer are in fluid communication with each other.

25. The mobile device of claim 23 further comprising:
at least one wire bond, wherein at least one wire bond that is encapsulated within the body.

26. The mobile device of claim 23, wherein the body comprises a substrate and an upper portion positioned adjacent to the substrate, and wherein the upper portion of the body has an opening adjacent to the chemochromic layer.

27. The mobile device of claim 21, wherein the housing comprises a movable protector and wherein the movable protector covers the opening in a first position and exposes the opening in a second position.

28. A sensing device for detecting one or more analytes, the sensing device comprising:
a body;
a sensor die disposed on a surface of the body;
a substantially transparent layer disposed on the body such that the sensor die is positioned between the surface of the body and the substantially transparent layer;
an externally exposed surface of the substantially transparent layer; and
a chemochromic layer disposed on the externally exposed surface of the substantially transparent layer, wherein the chemochromic layer exhibits a first color in a first state, and a second color in a second state when exposed to a predetermined analyte, and wherein the sensor die is configured to detect the change in color of the chemochromic layer.

29. The sensing device of claim 28, wherein the externally exposed surface of the transparent layer is sufficiently planar to facilitate contact between the sensing device and the predetermined analyte.

30. The sensing device of claim 28, wherein the chemochromic layer comprises a first chemochromic material and a second chemochromic material, wherein the first chemochromic material and the second chemochromic material exhibits different colors in response to different analytes.
